(19) United States
(12) Patent Application Publication
(10) Pub. No.: US 2009/0306466 A1
(43) Pub. Date: Dec. 10, 2009

(54) DEVICE

(76) Inventors: Frank BONADIO, Bray (IE); Trevor VAUGH, Birr (IE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/479,314

(22) Filed: Jun. 5, 2009

Related U.S. Application Data
(60) Provisional application No. 61/059,379, filed on Jun. 6, 2008.

(51) Int. Cl. A61B 1/32

(52) U.S. Cl. 600/37; 600/235

(57) ABSTRACT

A holding device is used during a laparoscopic surgery to hold an internal organ such as a gall bladder in a desired position in an insufflated abdominal cavity while maintaining pneumopertioneum. The device is used in association with an instrument which is used to introduce the device through an access port in the abdominal wall 6 of a patient. The instrument may be a grasper having gripping jaws which are used for manipulating the device, in use. The device may be used to retract an internal organ out of the surgical filed to allow more ready access to other organs or regions of interest. The device comprises a first gripping means in the form of hooks at one end for engaging an internal organ. The device also has a second gripping means provided by hooks which are engaged in the interior of the abdominal wall.

This application claims the benefit of U.S. Provisional Application No. 61/059,379 filed Jun. 6, 2008. The content of this provisional application is incorporated herein by reference.

### INTRODUCTION

This invention relates to an apparatus and a method of performing a laparoscopic procedure.

### STATEMENTS OF INVENTION

According to the invention there is provided a device for holding an internal organ in a desired position during a procedure, the device comprising first gripping means for gripping an organ or the like and second gripping means for mounting the device to a desired location.

In one embodiment the first gripping means comprises at least one hook for engaging an internal organ.

In one embodiment the gripping means is adapted to engage the wall of the organ without penetration through the wall.

In one case the second gripping means comprises at least one hook for mounting the device to a desired location.

The device may be mounted to an abdominal wall, in use. The device may be mounted to the interior of an abdominal wall.

In one embodiment the device or a portion thereof is movable from a first configuration in which at least one of the gripping means is inactive to an active gripping configuration.

In one case the device is movable from an inactive configuration to the active configuration for the second gripping means.

The device may comprise a movable section. The movable section may be operable by an instrument to move the gripping means from the inactive configuration to the active configuration.

In one case the movable section of the device comprises engagement means for engagement with an instrument.

The device may comprise a receiver for engagement with an instrument.

In one embodiment the device is at least partially flexible and/or malleable.

The invention also provides a method of performing a surgical procedure utilising the device of the invention.

In one embodiment the method is a method for holding an internal organ in a desired position during a surgical procedure comprising the steps of:—
providing a device having a first gripping means and a second gripping means;
introducing the device into the body;
gripping an organ using the first gripping means of the device;
moving the organ to expose a surgical field; and
using the second gripping means of the device to mount the device to a desired location.

In one case the device comprises a movable section and the method comprises manipulating the movable section to mount the device to a desired location.

The method may comprise engaging the movable section with an instrument and manipulating the device using the instrument.

In one case the method comprises a method of performing a laparoscopic surgical procedure. The method may comprise performing a cholecystectomy procedure.

### BRIEF DESCRIPTION OF THE DRAWINGS

The invention will be more clearly understood from the following description of some embodiments thereof, given by way of example only, with reference to the accompanying drawings, in which:—

FIGS. 1 to 4 are side views of an internal organ holding device according to the invention in different configuration of use;

FIG. 5 is an isometric view of the internal organ holding device in one configuration of use;

FIG. 6 is an isometric view of the device of FIGS. 1 to 5, in use;

FIGS. 7 to 13 are diagrams illustrating various steps involved in using the device of FIGS. 1 to 6;

FIG. 14 is another isometric view of the device;

FIG. 15 is an isometric view of another device of the invention;

FIG. 16 is an isometric view of alternative gripping features for the device;

FIG. 17 is a side view of the gripping features in use;

FIG. 18 is an isometric view of a further organ holding device according to the invention; and

FIG. 19 is an isometric view of the device of FIG. 18, in use.

### DETAILED DESCRIPTION

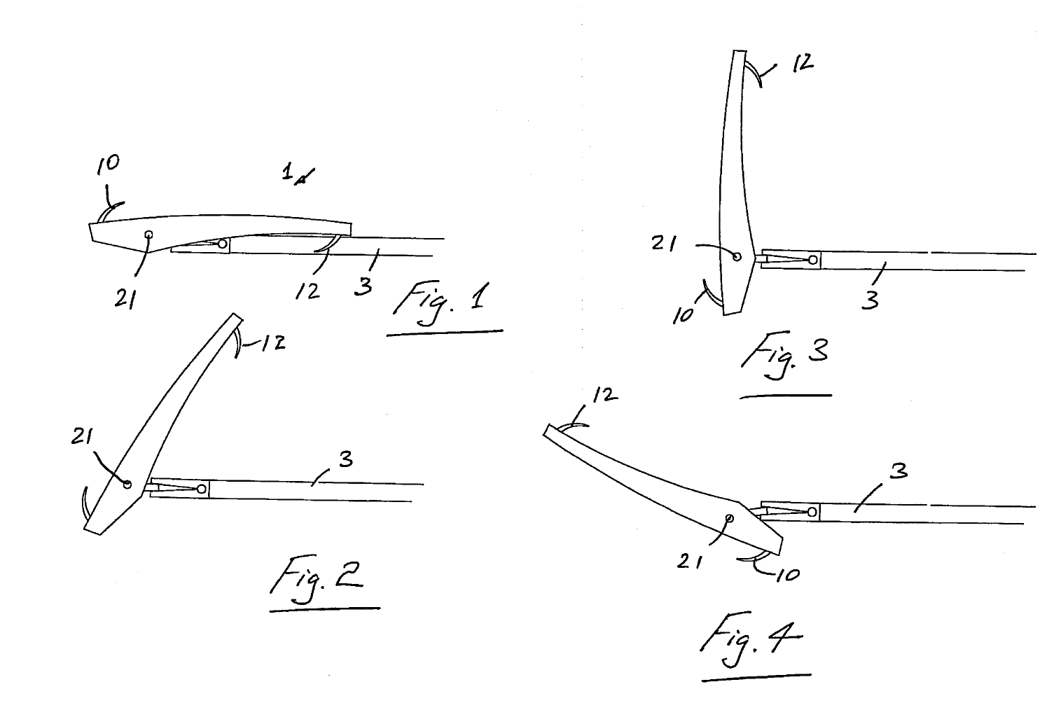

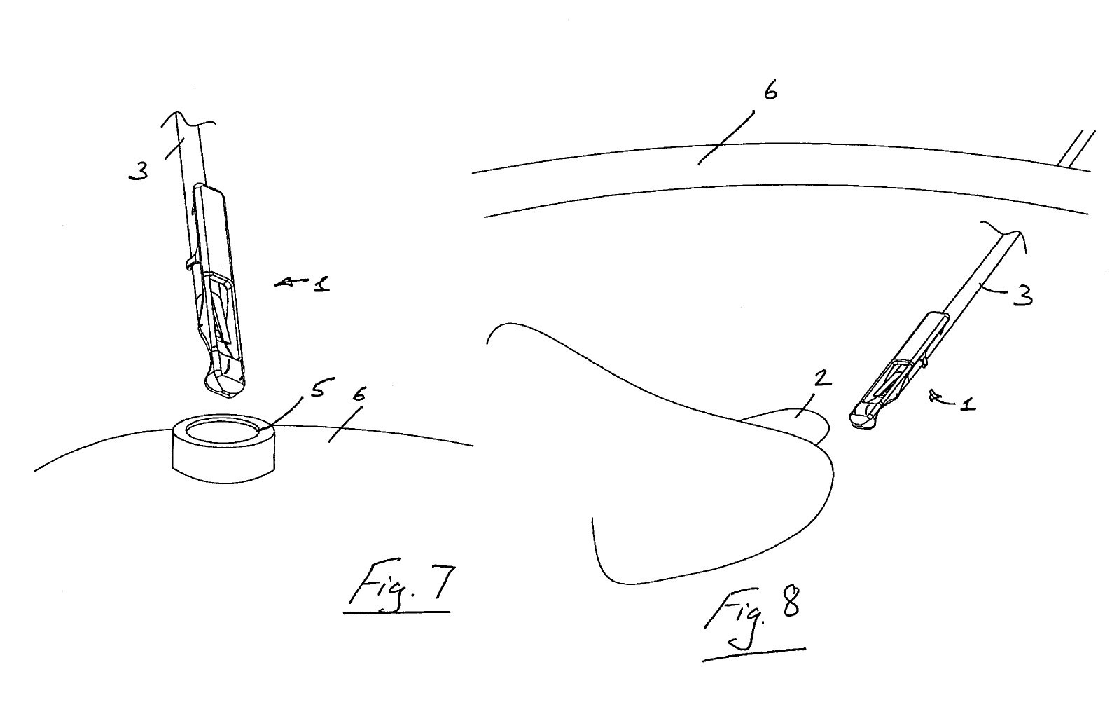

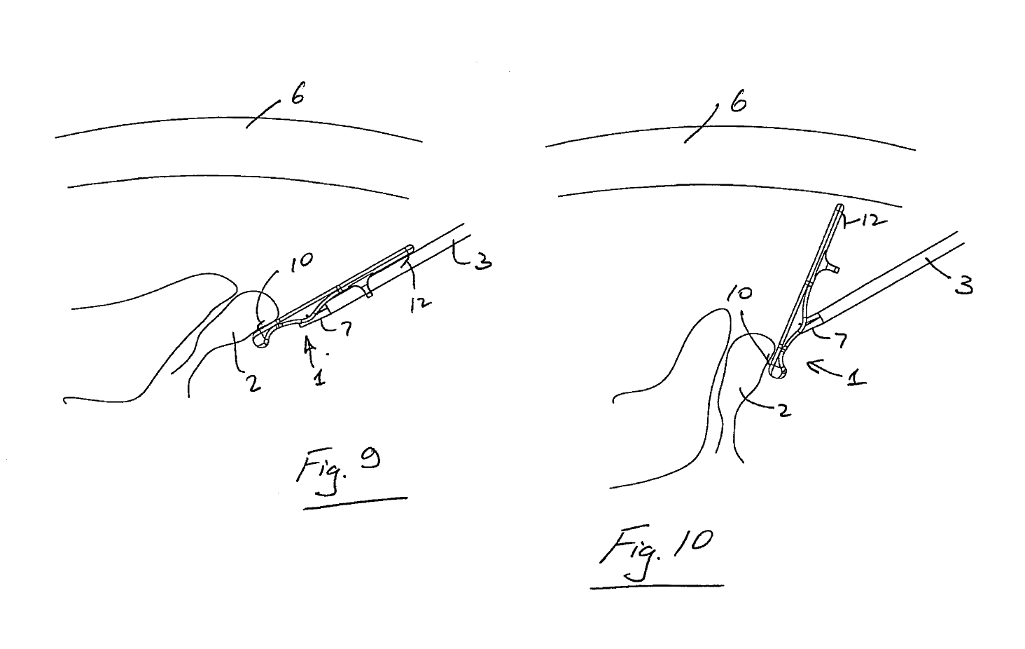

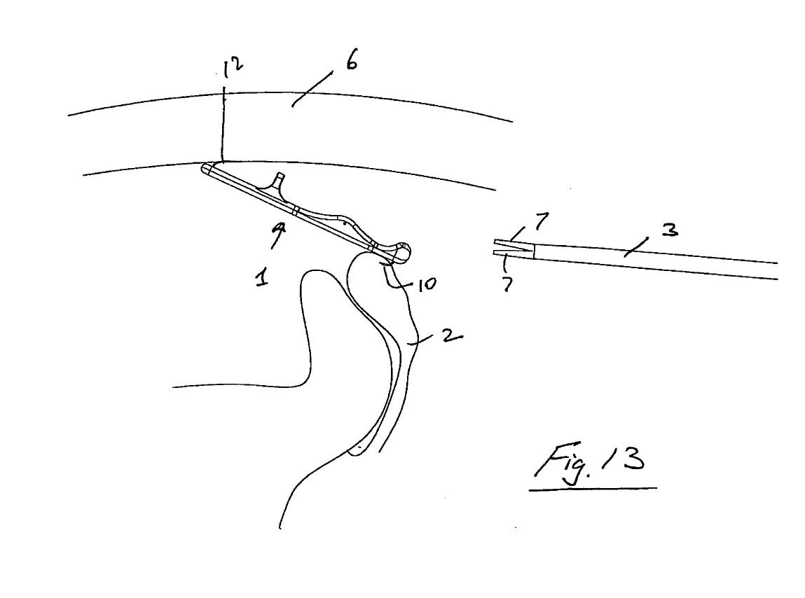

Referring to the drawings, and initially to FIGS. 1 to 14 thereof, there is illustrated a holding device 1 according to the invention. The device 1 is suitable for use during a procedure such as during laparoscopic surgery to hold an internal organ such as a gall bladder 2 in a desired position, for example in a desired position in an insufflated abdominal cavity while maintaining pneumoperitoneum.

In this case the device 1 is used in association with an instrument 3 which is used to introduce the device 1 through an access port 5 in the abdominal wall 6 of a patient. The access port may be of the type described in our US2009-0036745A, the content of which is incorporated herein by reference.

The instrument 3 is in this case a grasper having gripping jaws 7 which are used for manipulating the device, in use. The device may be used for example to retract an internal organ out of the surgical field to allow more ready access to other organs or regions of interest.

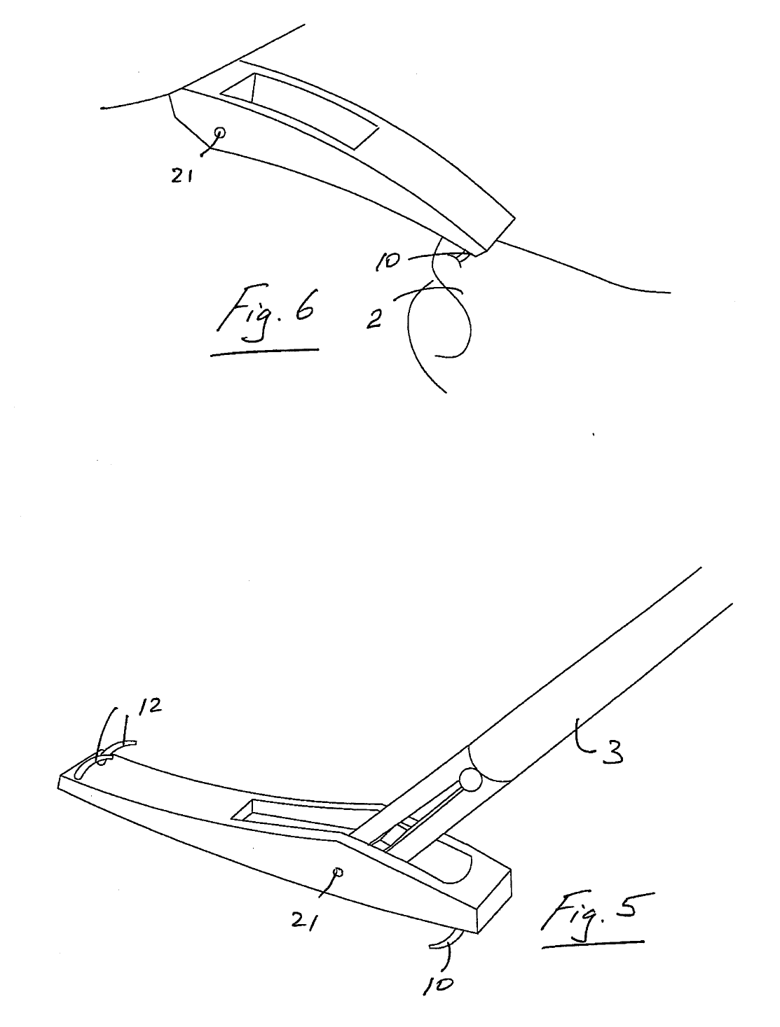

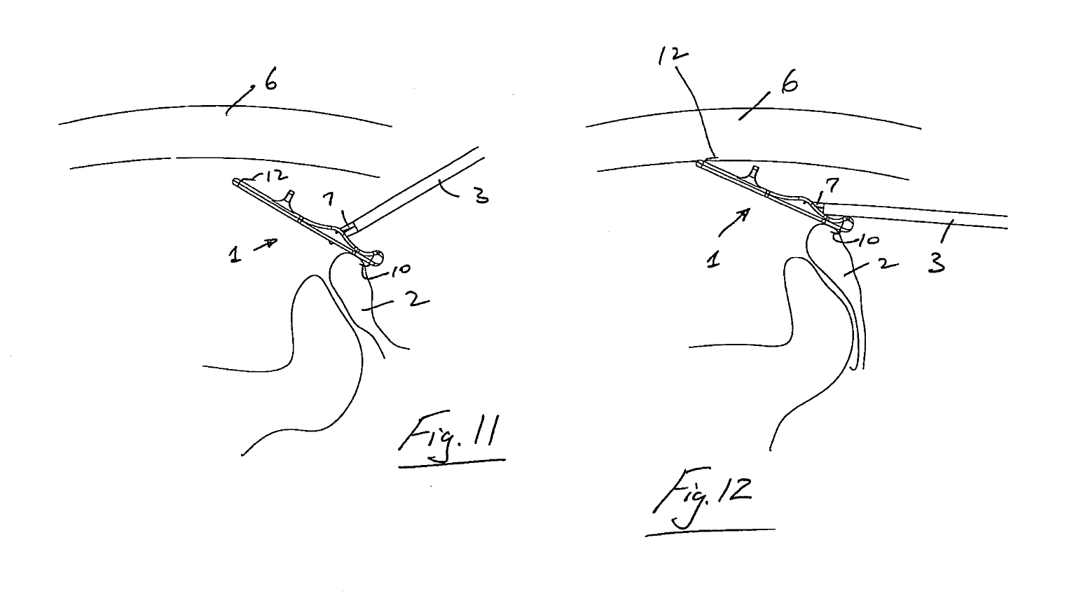

The device 1 comprises a first gripping means in the form of hooks 10 at one end for engaging an internal organ as shown especially in FIGS. 3 and 6 to 9. The device also has a second gripping means provided by hooks 12 which are used to retain the device in a desired position, in this case the hooks 12 are engaged in the interior of the abdominal wall 6 as illustrated especially in FIGS. 9 and 10.

The device 1 has a movable portion 20 which in this case is pivotally mounted about a pivot 21 for movement from a low profile insertion configuration as illustrated in FIG. 1 to a holding configuration as illustrated in FIG. 4. The movable portion 20 in this case has ridges for ease of gripping by the jaws 7 of the grasper 3. The grasper 3 is manipulated externally to cause the device to pivot relative to the instrument 3 which allows the hooks 12 to be readily manipulated into a position engaging the abdominal wall.

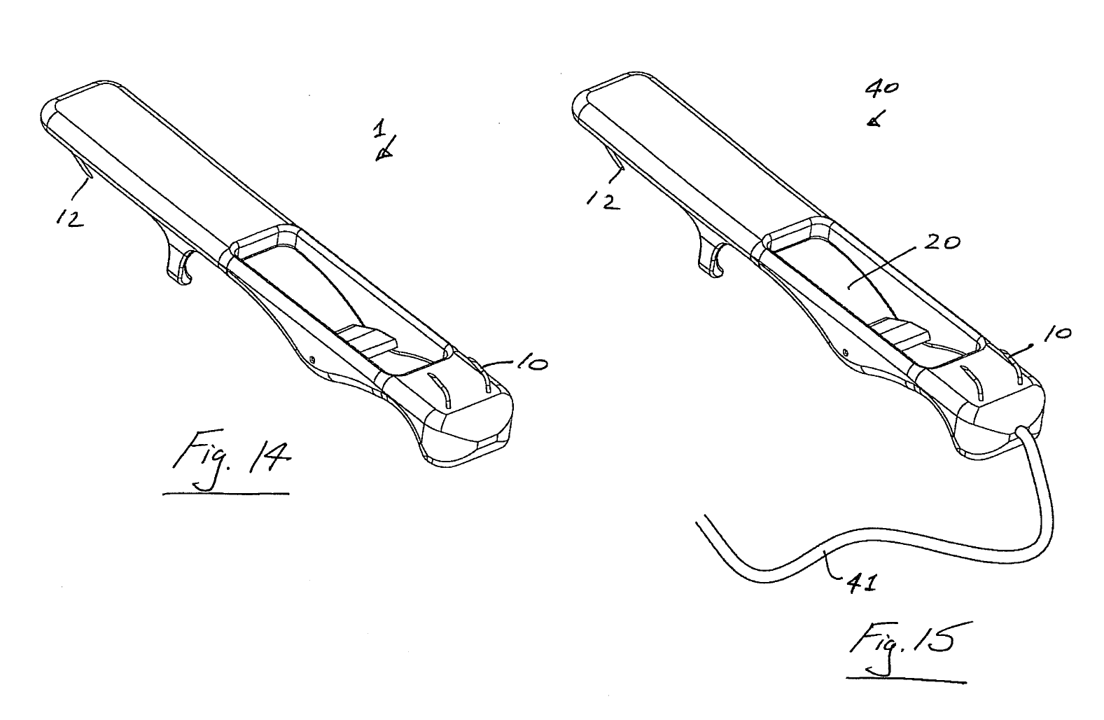

In use, the device 1 is mounted to an instrument 3 with the jaws 7 engaging the movable portion 20. Initially the device is in a low profile insertion configuration (FIG. 1) which allows the surgeon to readily introduce the device into the abdominal cavity through an access port. The surgeon manipulates the device such that the first hooks 10 engage with the internal organ 2 which is to be manipulated. The surgeon then moves the instrument 3 so that the device pivots and the second hooks 12 are engaged with the inside of the abdominal wall. In this way a surgeon retracts the internal organ to expose a wider surgical field. When the device 1 is in place the instrument 3 can be removed [FIG. 13].

The device greatly facilitates efficient surgical procedures as a surgeon is free to use several instruments to continue the procedure.

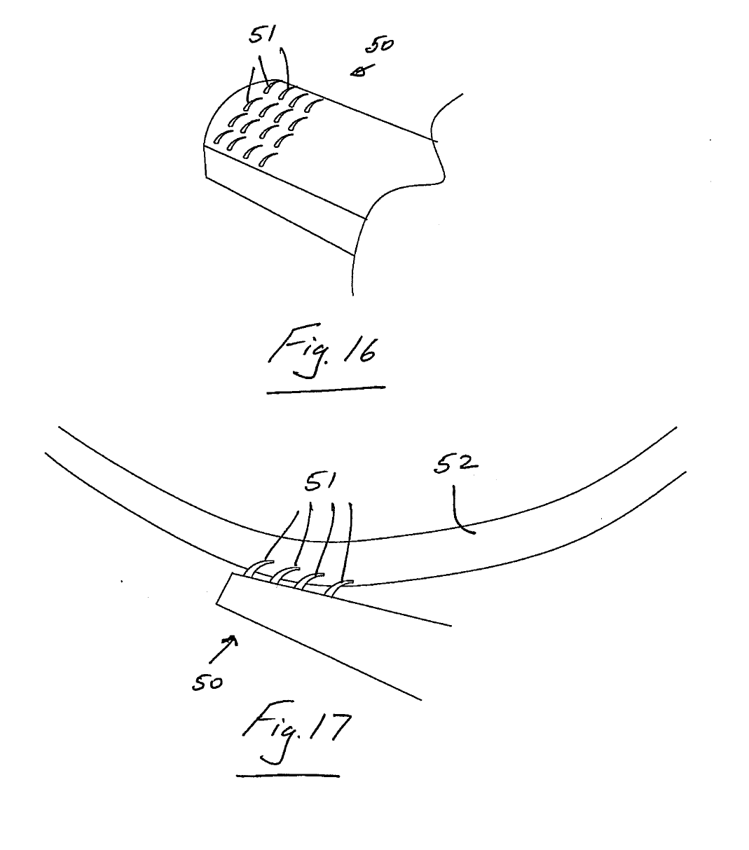

Referring to FIG. 15 there is illustrated another device 40 according to the invention which is similar to the device of FIGS. 1 to 14 and like parts are assigned the same reference numerals. In this case the device is also provided with a suction line 41.

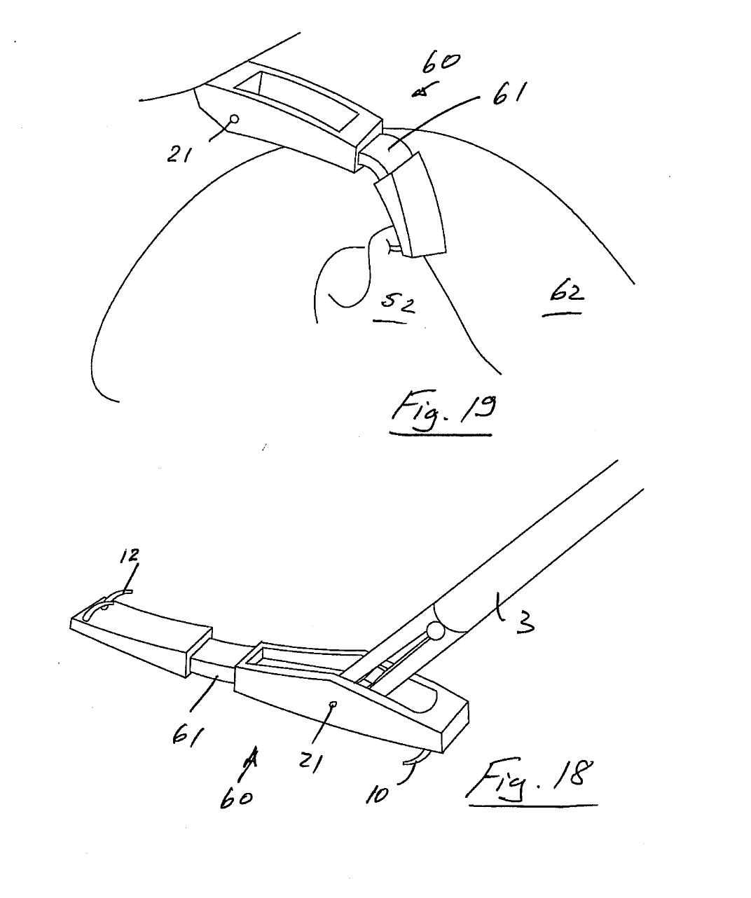

Referring to FIGS. 16 and 17 there is illustrated alternative gripping features 50 for the device. In this case there is an array of small barbs 51 which are long enough to allow the wall of the organ such as a gall bladder 52 to be engaged without penetrating the wall of the gall bladder and hence avoiding possible leakage from the organ such as a gall bladder 52.

Referring to FIGS. 18 and 19 there is illustrated an alternative device 60 according to the invention which is similar to those described above and like parts are assigned the same reference numerals. In this case the device has a flexible section 61 which facilitates flexing around an organ such as the liver 62 to achieve improved retraction of the gall bladder 52 to allow access for laparascopic surgical procedures. The section 61 may be malleable and/or manipulated by the surgeon so that when flexed it at least retains the curved shape. It will be appreciated that the grip features 51 described with reference to FIGS. 16 and 17 may also be used in this version of the device.

The invention is not limited to the embodiments hereinbefore described, with reference to the accompanying drawings, which may be varied in construction and detail.

1. A device for holding an internal organ in a desired position during a procedure, the device comprising first gripping means for gripping an organ and second gripping means for mounting the device to a desired location.

2. A device as claimed in claim 1 wherein the first gripping means comprises at least one hook for engaging an internal organ.

3. A device as claimed in claim 2 wherein the gripping means is adapted to engage the wall of the organ without penetration through the wall.

4. A device as claimed in claim 1 wherein the second gripping means comprises at least one hook for mounting the device to a desired location.

5. A device as claimed in claim 1 wherein the device is mountable to an abdominal wall.

6. A device as claimed in claim 5 wherein the device is mountable to the interior of an abdominal wall.

7. A device as claimed in claim 1 wherein at least one of the gripping means is movable from an inactive delivery configuration to an active gripping configuration.

8. A device as claimed in claim 7 wherein the second gripping means is movable from the inactive configuration to the active configuration.

9. A device as claimed in claim 7 wherein the device comprises a movable section.

10. A device as claimed in claim 9 wherein the movable section is operable by an instrument to move the section from an inactive configuration to the active configuration for the gripping means.

11. A device as claimed in claim 10 wherein the movable section of the device comprises engagement means for engagement with an instrument.

12. A device as claimed in claim 1 comprising a receiver for engagement with an instrument.

13. A device as claimed in claim 1 wherein the device is at least partially flexible.

14. A device as claimed in claim 1 wherein the device is at least partially malleable.

15. A device as claimed in claim 1 having a suction line.

16. A method for holding an internal organ in a desired position during a surgical procedure comprising the steps of:—
providing a device having a first gripping means and a second gripping means;
introducing the device into the body;
gripping an organ using the first gripping means of the device;
moving the organ to expose a surgical field; and
using the second gripping means of the device to mount the device to a desired location.

17. A method as claimed in claim 16 wherein the device comprises a movable section and the method comprises manipulating the movable section to mount the device to a desired location.

18. A method as claimed in claim 17 comprising engaging the movable section with an instrument and manipulating the device using the instrument.

19. A method as claimed in claim 16 wherein the surgical procedure is a laparoscopic procedure.

20. A method as claimed in claim 16 wherein the internal organ is the gall bladder.

21. A method as claimed in claim 16 wherein the surgical procedure is a cholecystectomy procedure.
